(19) United States
(12) Patent Application Publication
(10) Pub. No.: US 2004/0116495 A1
(43) Pub. Date: Jun. 17, 2004

(54) Compounds and methods

(76) Inventors: Joseph P. Marino Jr., King of Prussia, PA (US); Scott K. Thompson, King of Prussia, PA (US); Daniel Frank Veber, Ambler, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/473,160

(22) PCT Filed: Mar. 28, 2002
(86) PCT No.: PCT/US02/09660

(30) Foreign Application Priority Data
Mar. 29, 2001 (US) 60279558

(51) Int. Cl. A61K 31/4164

(52) U.S. Cl. 514/398

(57) ABSTRACT

Compounds of this invention are non-peptide, reversible inhibitors of type 2 methionine aminopeptidase, useful in treating conditions mediated by angiogenesis, such as cancer, haemangioma, proliferative retinopathy, rheumatoid arthritis, atherosclerotic neovascularization, psoriasis, ocular neovascularization and obesity.

### FIELD OF THE INVENTION

Compounds of this invention are non-peptide, reversible inhibitors of type 2 methionine aminopeptidase, useful in treating conditions mediated by angiogenesis, such as cancer, haemangioma, proliferative retinopathy, rheumatoid arthritis, atherosclerotic neovascularization, psoriasis, ocular neovascularization and obesity.

### BACKGROUND OF THE INVENTION

In 1974, Folkman proposed that for tumors to grow beyond a critical size and to spread to form metastases, they must recruit endothelial cells from the surrounding stroma to form their own endogenous microcirculation in a process termed angiogenesis (Folkman J. (1974) Adv Cancer Res. 19; 331). The new blood vessels induced by tumor cells as their life-line of oxygen and nutrients also provide exits for cancer cells to spread to other parts of the body. Inhibition of this process has been shown to effectively stop the proliferation and metastasis of solid tumors. A drug that specifically inhibits this process is known as an angiogenesis inhibitor.

Having emerged as a promising new strategy for the treatment of cancer, the anti-angiogenesis therapy (“indirect attack”) has several advantages over the “direct attack” strategies. All the “direct attack” approaches such as using DNA damaging drugs, antimetabolites, attacking the RAS pathway, restoring p53, activating death programs, using aggressive T-cells, injecting monoclonal antibodies and inhibiting telomerase, etc., inevitably result in the selection of resistant tumor cells. Targeting the endothelial compartment of tumors as in the “indirect attack”, however, should avoid the resistance problem because endothelial cells do not exhibit the same degree of genomic instability as tumor cells. Moreover, anti-angiogenic therapy generally has low toxicity due to the fact that normal endothelial cells are relatively quiescent in the body and exhibit an extremely long turnover. Finally since the “indirect attack” and “direct attack” target different cell types, there is a great potential for a more effective combination therapy.

More than 300 angiogenesis inhibitors have been discovered, of which about 31 agents are currently being tested in human trials in treatment of cancers (Thompson, et al., (1999) J Pathol 187, 503). TNP-470, a semisynthetic derivative of fumagillin of Aspergillus fuigatus, is among the most potent inhibitors of angiogenesis. It acts by directly inhibiting endothelial cell growth and migration in vitro and in vivo (Ingber et al. (1990) Nature 348, 555). Fumagillin and TNP-470, have been shown to inhibit type 2 methionine aminopeptidase (hereinafter MetAP2) by irreversibly modifying its active site. The biochemical activity of fumagillin analogs has been shown to correlate to their inhibitory effect on the proliferation of human umbillical vein endothelial cells (HUVEC). Although the mechanism of the selective action of fumagillin and related compounds on MetAP2-mediated endothelial cell cytostatic effect has not yet been established, possible roles of MetAP2 in cell proliferation have been suggested.

First, hMetAP-2-catalyzed cleavage of the initiator methionine of proteins could be essential for releasing many proteins that, after myristoylation, function as important signaling cellular factors involved in cell proliferation. Proteins known to be myristoylated include the src family tyrosine kinases, the small GTPase ARF, the HIV protein nef and the α subunit of heterotrimeric G proteins. A recently published study has shown that the myristoylation of nitric oxide synthase, a membrane protein involved in cell apoptosis, was blocked by fumagillin (Yoshida, et al: (1998) Cancer Res. 58(16), 3751). This is proposed to be an indirect outcome of inhibition of MetAP2-catalyzed release of the glycine-terminal myristoylation substrate. Alternatively, MetAP enzymes are known to be important to the stability of proteins in vivo according to the “N-end rule” which suggests increased stability of methionine-cleaved proteins relative to their N-terminal methionine precursors (Varshavsky, A (1996) Proc. Natl. Acad. Sci. U.S.A. 93, 12142). Inhibition of hMetAP2 could result in abnormal presence or absence of some cellular proteins critical to the cell cycle.

Methionine aminopeptidases (MetAP) are ubiquitously distributed in all living organisms. They catalyze the removal of the initiator methionine from newly translated polypeptides using divalent metal ions as cofactors. Two distantly related MetAP enzymes, type 1 and type 2, are found in eukaryotes, which at least in yeast, are both required for normal growth; whereas only one single MetAP is found in eubacteria (type 1) and archaebacteria (type 2). The N-terminal extension region distinguishes the methionine aminopeptidases in eukaryotes from those in procaryotes. A 64-amino acid sequence insertion (from residues 381 to 444 in hMetAP2) in the catalytic C-terminal domain distinguishes the MetAP-2 family from the MetAP-1 family. Despite the difference in the gene structure, all MetAP enzymes appear to share a highly conserved catalytic scaffold termed “pita-bread” fold (Bazan, et al. (1994) Proc. Natl. Acad. Sci. U.S.A. 91, 2473), which contains six strictly conserved residues implicated in the coordination of the metal cofactors.

Mammalian type 2 methionine aminopeptidase has been identified as a bifunctional protein implicated by its ability to catalyze the cleavage of N-terminal methionine from nascent polypeptides (Bradshaw, et al (1998) Trends Biochem. Sci. 23, 263) and to associate with eukaryotic initiation factor 2α (eIF-2α) to prevent its phosphorylation (Ray, et al. (1992) Proc. Natl. Acad. Sci. U.S.A. 89, 539). Both the genes of human and rat MetAP2 were cloned and have shown 92% sequence identity (Wu, et al. (1993) J. Biol. Chem. 268, 10796; Li, X. & Chang, Y.-H. (1996) Biochem. & Biophys. Res. Comm. 227, 152). The N-terminal extension in these enzymes is highly charged and consists of two basic polylysine blocks and one aspartic acid block, which has been speculated to be involved in the binding of eIF-2α (Gupta, et al. (1993) in Translational Regulation of Gene Expression 2 (Ilan, J., Ed.), pp. 405-431, Plenum Press, New York).

The anti-angiogenic compounds, fumagillin and its analogs, have been shown to specifically block the exo-aminopeptidase activity of hMetAP2 without interfering with the formation of the hMetAP2: eIF2α complex (Griffith, et al., (1997) Chem Biol. 4, 461; Sin, et al. (1997) Proc. Natl. Acad. Sci. U.S.A. 94, 6099). Fumagillin and its analogs inactivate the enzymatic activity of hMetAP2 with a high specificity, which is underscored by the lack of effect of these compounds on the closely related type 1 methionine aminopeptidase (MetAP1) both in vitro and in vivo in yeast (Griffith, et al., (1997) Chem Biol. 4, 461; Sin, et al. (1997) Proc. Natl. Acad. Sci. U.S.A. 94, 6099). The extremely high potency (IC50<1 nM) of these inhibitors appears to be due to the irreversible modification of the active site residue, His231, of hMetAP2 (Liu, et al. (1998) Science 282, 1324). Disturbance of MetAP2 activity in vivo impairs the normal growth of yeast (Griffith, et al., (1997) Chem. Biol. 4, 461; Sin, et al. (1997) Proc. Natl. Acad. Sci. U.S.A. 94, 6099; In-house data) as well as Drosophila (Cutforth & Gaul (1999) Mech. Dev. 82, 23). Most significantly, there appears to be a clear correlation between the inhibition effect of fumagillin related compounds against the enzymatic activity of hMetAP2 in vitro and the suppression effect of these compounds against tumor-induced angiogenesis in vivo (Griffith, et al., (1997) Chem. Biol. 4, 461).

Cancer is the second leading cause of death in the U.S., exceeded only by heart disease. Despite recent successes in therapy against some forms of neoplastic disease, other forms continue to be refractory to treatment. Thus, cancer remains a leading cause of death and morbidity in the United States and elsewhere (Bailar and Gomik (1997) N Engl J Med 336, 1569). Inhibition of hMetAP2 provides a promising mechanism for the development of novel anti-angiogenic agents in the treatment of cancers. It has now been discovered that compounds of formulae (1) and (IA) are effective inhibitors of hMetAP2, and thus would be useful in treating conditions mediated by hMetAP2.

### SUMMARY OF THE INVENTION

In one aspect, the present invention is to a compound of formula (I) or formula (IA), or a pharmaceutically active salt or solvate thereof, and its use in treating conditions mediated by angiogenesis, such as cancer, haemangioma, proliferative retinopathy, rheumatoid arthritis, atherosclerotic neovascularization, psoriasis, ocular neovascularization and obesity:

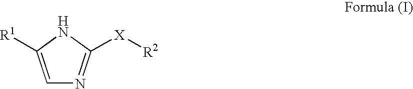

wherein:

X is S or O;

R¹ is optionally substituted C₁₋₆alkyl, C₂₋₆alkenyl, C₂₋₆alkynyl, optionally substituted Ar—C₀₋₆alkyl-, optionally substituted Het-C₀₋₆alkyl-, optionally substituted C₃₋₇cycloalkyl-C₀₋₆alkyl-; provided that when R¹ is optionally substituted Het-C₁₋₄alkyl-, and Het is indolyl, benzofuranyl, benzothienyl, benzisoxazolyl, benzisothiazolyl, benzopyrazolyl, or pyrrolo[2,3-c]pyridinyl then the optional substituent is not —(CH₂)₁₋₅CHR^{I}NR^{II}R^{III}, or the optional substitutent is not a 4- to 6-membered heterocycle which contains one nitrogen; or provided that when R¹ is Ar—C₁₋₂alkyl-, Ar may not be phenyl optionally substituted at the meta or para position with —CN, —C(═NR)NR′R″, —NHC(═NR)R′R″—NRC═NR, or —CONRR′, wherein R, R′ and R″ are independently H, C₁₋₆alkyl, C₂₋₆alkenyl, C₂₋₆alkynyl, Ar—C₀₋₆alkyl-, Het-C₀₋₆alkyl-, or C₃₋₇cycloalkyl-C₀₋₆alkyl-; or provided that when R¹ is optionally substituted Ar—C₁alkyl-, the optional substituents may not both be —OH and phenyl or a saturated 6-membered ring containing one nitrogen;

R^{I} is H or C₁₋₆alkyl;

R^{II} and R^{III} are independently H, C₁₋₆alkyl, or together with the nitrogen to which they are attached form a 4-6 membered heterocyclic ring which optionally contains one or more additional heteroatoms selected from N, O, and S; and

R² is optionally substituted C₁₋₆alkyl, C₃₋₆alkenyl, C₃₋₆alkynyl, optionally substituted Ar—C₀₋₆alkyl-, optionally substituted Het-C₀₋₆alkyl-, or optionally substituted C₃₋₇cycloalkyl-C₀₋₆alkyl-; and provided that when R¹ is optionally substituted Ar—C₀alkyl-, or optionally substituted C₅₋₆cycloalkyl-C₀alkyl- then R² cannot be C₁₋₆alkyl; provided that when R¹ is optionally substituted Ar—C₀₋₁alkyl-, or C₅₋₆cycloalkyl-C₀alkyl- then R² cannot be imidazolyl-C₂₋₃alkyl-, where the alkyl chain is directly attached to moiety X; or provided that the compound is not 2-[[(4-phenyl-1H-imidazol-2-yl)thio]methyl]-pyridinyl, 4-phenyl-2-(benzylthio)-1H-imidazole, 4-phenyl-2-(4-chloro-benzylthio)-1H-imidazole, 4-phenyl-2-(2-methylamino-benzylthio)-1H-imidazole, 4-phenyl-2-(2-propenylthio)-1H-imidazole, 2-[[4-(3-thienyl)-1H-imidazol-2-yl]thio]-hexanoic acid, 4-phenyl-2-(phenylthio)-1H-imidazole, 4-cyclohexyl-2-[(2-methyl-2-propenyl)thio]-1H-imidazole, 4-(1-methylcyclohexyl)-2-[(2-methyl-2-propenyl)thio]-1H-imidazole, 2,6-bis(1,1-dimethylethyl)-4-[2-[(phenylmethyl)thio]-1H-imidazole, 2-[[[4-(4-methoxyphenyl)-1H-imidazol-2-yl]thio]methyl]-pyridinyl, 2-[[[4-(4-bromophenyl)-1H-imidazol-2-yl]thio]methyl]-pyridinyl, and 1-(cyclopropylamino)-3-[[4-(2-thienyl)-1H-imidazol-2-yl]oxy]-2-propanol dihydrochloride.

In a second aspect, the present invention is to a method of treating conditions mediated by angiogenesis, such as cancer, haemangioma, proliferative retinopathy, rheumatoid arthritis, atherosclerotic neovascularization, psoriasis, ocular neovascularization and obesity by administering a compound of formula (IA), or a pharmaceutically acceptable salt or solvate thereof:

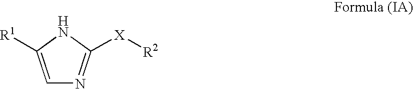

wherein,

X is S or O;

R¹ is optionally substituted C₁₋₆alkyl, C₂₋₆alkenyl, C₂₋₆alkynyl, optionally substituted Ar—C₀₋₆alkyl-, optionally substituted Het-C₀₋₆alkyl-, or optionally substituted C₃₋₇cycloalkyl-C₀₋₆alkyl-; and

R² is optionally substituted C₁₋₆alkyl, C₃₋₆alkenyl, C₃₋₆alkynyl, optionally substituted Ar—C₀₋₆alkyl-, optionally substituted Het-C₀₋₆alkyl-, or optionally substituted C₃₋₇cycloalkyl-C₀₋₆alkyl-.

In another aspect, the present invention is to a method of inhibiting MetAP2 in the treatment of angiogenesis-mediated diseases, all in mammals, preferably humans, comprising administering to such mammal in need thereof, a compound of formula (IA), or a pharmaceutically active salt or solvate thereof.

In yet another aspect, the present invention is to pharmaceutical compositions comprising a compound of formula (I) or formula (IA), including a pharmaceutically acceptable salt or solvate thereof, and a pharmaceutically acceptable carrier therefor. In particular, the pharmaceutical compositions of the present invention are used for treating MetAP2-mediated diseases.

### DETAILED DESCRIPTION OF THE INVENTION

It has now been discovered that substituted imidazoles of formula (1) are inhibitors of MetAP2. It has also now been discovered that selective inhibition of MetAP2 enzyme mechanisms by treatment with the inhibitors of formula (I) or formula (IA), or a pharmaceutically acceptable salt or solvate thereof, represents a novel therapeutic and preventative approach to the treatment of a variety of disease states, including, but not limited to, cancer, haemangioma, proliferative retinopathy, rheumatoid arthritis, atherosclerotic neovascularization, psoriasis, ocular neovascularization and obesity.

The term “C₁₋₆alkyl” as used herein at all occurrences means a substituted and unsubstituted, straight or branched chain radical of 1 to 6 carbon atoms, unless the chain length is limited thereto, including, but not limited to methyl, ethyl, n-propyl, isopropyl, n-butyl, isobutyl and t-butyl, pentyl, n-pentyl, isopentyl, neopentyl and hexyl and the simple aliphatic isomers thereof.

Any C₁₋₆alkyl group may be optionally substituted independently by one or more of —OR³, —R³, —NR³R⁴. C₀alkyl means that no alkyl group is present in the moiety. Thus, Ar—C₀alkyl- is equivalent to Ar.

As used herein at all occurrences, substituents R³, R⁴, and R⁵ are independently defined as C₂₋₆alkyl, C₃₋₆alkenyl, C₃₋₆alkynyl, Ar—C₀₋₆alkyl-, Het-C₀₋₆alkyl-, or C₃₋₇cycloalkyl-C₀₋₆alkyl-.

The term “C₃₋₇cycloalkyl” as used herein at all occurrences means substituted or unsubstituted cyclic radicals having 3 to 7 carbons, including but not limited to cyclopropyl, cyclopentyl, cyclohexyl and cycloheptyl radicals. Any C₁₋₆cycloalkyl group may be optionally substituted independently by one or more of —OR³, —R³, —NR³R⁴.

The term “C₂₋₆alkenyl” as used herein at all occurrences means an alkyl group of 2 to 6 carbons, unless the chain length is limited thereto, wherein a carbon-carbon single bond thereof is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included within the scope of this invention.

Any C₂₋₆alkenyl group may be optionally substituted independently by one or more of Ph-C₀₋₆alkyl-, Het′-C₀₋₆ alkyl-, C₁₋₆alkyl, C₁₋₆alkoxy-, C₁₋₆alkyl-S—, Ph-C₀₋₆alkoxy-, Het′-C₀₋₆alkoxy-, —OH, —NR³R⁴, Het′-S—C₀₋₆alkyl-, —(CH₂)₁₋₆OH, —(CH₂)₁₋₆NR³R⁴, —O(CH₂)₁₋₆NR³R⁴, —(CH₂)₀₋₆CO₂R⁵, —O(CH₂)₁₋₆CO₂R⁵, —(CH₂)₁₋₆SO₂R⁵, —CF₃, —OCF₃ or halogen.

The term “C₂₋₆alkynyl” as used herein at all occurrences means an alkyl group of 2 to 6 carbons, unless the chain length is limited thereto, wherein one carbon-carbon single bond is replaced by a carbon-carbon triple bond. C₂₋₆ alkynyl includes 1-propyne, 2-propyne, 1-butyne, 2-butyne, 3-butyne and the simple isomers of pentyne and hexyne.

Any C₂₋₆alkynyl group may be optionally substituted independently by one or more of Ph-C₀₋₆alkyl-, Het′-C₀₋₆ alkyl-, C₁₋₆alkyl, C₁₋₆alkoxy-, C₁₋₆alkyl—S—, Ph-C₀₋₆alkoxy-, Het′-C₀₋₆alkoxy-, —OH, —NR³R⁴, Het′—S—C₀₋₆alkyl-, —(CH₂)₁₋₆OH, —(CH₂)₁₋₆NR³R⁴, —O(CH₂)₁₋₆NR³R⁴, —(CH₂)₀₋₆CO₂R⁵, —O(CH₂)₁₋₆CO₂R⁵, —(CH₂)₁₋₆SO₂R⁵, —CF₃, —OCF₃ or halogen.

The terms “Ar” or “aryl” as used herein interchangeably at all occurrences mean phenyl and naphthyl, optionally substituted by one or more of Ph-C₀₋₆alkyl-, Het′-C₀₋₆alkyl-, C₁₋₆alkyl, C₁₋₆alkoxy, C₁₋₆alkyl-S—, Ph-C₀₋₆alkoxy-, Het′-C₀₋₆alkoxy-, —OH, —NR³R⁴, Het′-S—C₀₋₆alkyl-, —(CH₂)₁₋₆OH, —(CH₂)₁₆NR³R⁴, —O(CH₂)₁₋₆NR³R⁴, —(CH₂)₀₋₆CO₂R⁵, —O(CH₂)₁₋₆CO₂R⁵, —(CH₂)₁₋₆SO₂R⁵, —CF₃, —OCF₃ or halogen; in addition, Ph may be optionally substituted with one or more of C₁₋₆alkyl, C₁₋₆alkoxy-, —OH, —(CH₂)₁₋₆NR³R⁴, —O(CH₂)₁₋₆NR³R⁴, —CO₂R⁵, CF₃, or halogen; Het′is defined as for Het, and may be optionally substituted by one or more of C₁₋₆alkyl, C₁₋₆alkoxy, —OH, (CH₂)₁₋₆NR³R⁴, —O(CH₂)₁₋₆NR³R⁴, —CO₂R⁵, —CF₃, or halogen; or two C₁₋₆alkyl or C₁₋₆alkoxy- groups may be combined to form a 5-7 membered, saturated or unsaturated ring, fused onto the Ar ring (e.g., to form a divalent alkylene or alkylene or alkylenedioxy moiety attached to adjacent positions on the Ar ring).

Suitably, for compounds of formula (I), when Ar is substituted by Ph or Het′, then Ph or Het′ are substituted with one or more of C₂₋₆alkyl, C₁₋₆alkoxy-, —(CH₂)₁₋₆NR³R⁴, —O(CH₂)₁₋₆NR³R⁴, —CO₂R⁵, —CF₃ or halogen.

The terms “Het” or “heterocyclic” as used herein interchangeably at all occurrences, mean a stable 5- to 7-membered monocyclic, a stable 7- to 10-membered bicyclic, or a stable 11- to 18-membered tricyclic heterocyclic ring, all of which are either saturated or unsaturated, and consist of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure.

It will be understood that Het may be optionally substituted with one or more of Ph-C₀₋₆alkyl-, Het′-C₀₋₆alkyl-, C₁₋₆alkyl, C₁₋₆alkoxy-, C₁₋₆alkyl-S—, Ph-C₀₋₆alkoxy-, Het′-C₀₋₆alkoxy-, —OH, —NR³R⁴, Het′-S—C₀₋₆alkyl-, —(CH₂)₁₋₆OH, (CH₂)₁₋₆NR³R⁴, —O(CH₂)₁₋₆NR³R⁴, —(CH₂)₀₋₆CO₂R⁵, —O(CH₂)₁₋₆CO₂R⁵, —(CH₂)₁₋₆SO₂R⁵, —CF₃, —OCF₃, —CN, or halogen; Ph may be optionally substituted with one or more of C₁₋₆alkyl, C₁₋₆alkoxy, —OH, —(CH₂)₁₋₆NR³R⁴, —O(CH₂)₁₋₆NR³R⁴, —CO₂R⁵, —CF₃, or halogen; and two C₁₋₆alkyl or C₁₋₆alkoxy groups may be combined to form a 5-7 membered ring, saturated or unsaturated, fused onto the Het ring (e.g., to form a divalent alkylene or alkylenedioxy moiety attached to adjacent positions on the Het ring). Preferred optional substituents on Het are C₁₋₆alkyl, C₁₋₆alkoxy-, C₁₋₆alkyl-S—, halogen, —CF₃, —OCF₃, —CN, or —NR³R⁴.

Het′ is defined as for Het and may be optionally substituted by one or more of C₁₋₆alkyl, C₁₋₆alkoxy-, —OH, —(CH₂)₁₆NR³R⁴, —O(CH₂)₁₋₆NR³R⁴, —CO₂R⁵, —CF₃, or halogen.

Examples of such heterocycles include, but are not limited to piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranylyl, benzothiophenyl, thiamorpholinyl sulfoxide, thiamorpholinyl sulfone, and oxadiazolyl, triazolyl, thiadiazolyl, oxadiazolyl, isoxazolyl, isothiazolyl, imidazolyl, pyridazinyl, pyrimidinyl and triazinyl which are available by routine chemical synthesis and are stable.

Compounds of this invention of formula (I), do not include compounds wherein when R² is optionally substituted Het-C₀alkyl-, Het is indolyl, benzofuranyl, benzothienyl, benzisoxazolyl, benzothiozole or benzopyrazolyl, and the optional substituent is —(CH₂)₁₋₅CHR^{I}NR^{II}R^{III}.

Further, it will be understood that when a moiety is “optionally substituted” the moiety may have one or more optional substituents, each optional substituent being independently selected.

The terms “hetero” or “heteroatom” as used herein interchangeably at all occurrences mean oxygen, nitrogen and sulfur.

The terms “halo” or “halogen” as used herein interchangeably at all occurrences mean F, Cl, Br, and I.

Here and throughout this application the term C₀ denotes the absence of the substituent group immediately following; for instance, in the moiety ArC₀₋₆alkyl-, when C is 0, the substituent is Ar, e.g., phenyl. Conversely, when the moiety ArC₀₋₆alkyl- is identified as a specific aromatic group, e.g., phenyl, it is understood that C is 0.

Suitably X is sulfur or oxygen. Preferably X is sulfur.

Suitably, R¹ is optionally substituted C₁₋₆alkyl, C₂₋₆alkenyl, C₂₋₆alkynyl, optionally substituted Ar—C₀₋₆alkyl-, optionally substituted Het-C₀₋₆alkyl-, or optionally substituted C₃₋₇cycloalkyl-C₀₋₆alkyl-. Preferably, R¹ is optionally substituted Ar—C₀₋₆alkyl-, Het-C₀₋₆alkyl-, or C₃₋₇cycloalkyl-C₀₋₆alkyl-. More preferably R¹ is optionally substituted Ar—C₁alkyl- (wherein the optional substituent is either in the ortho position or the para position), Het-C₁alkyl-, or C₅₋₆cycloalkyl-C₁alkyl-. Most preferably R¹ is optionally substituted Ar—C₁alkyl-, wherein the optional substituent is ortho C₁₋₆alkyl, preferably branched C₁₋₆alkyl, most preferably isopropyl.

Suitably, R² is optionally substituted C₁₋₆alkyl, C₃₋₆alkenyl, C₃₋₆alkynyl, optionally substituted Ar—C₀₋₆alkyl-, optionally substituted Het-C₀₋₆alkyl-, or optionally substituted C₃₋₇cycloalkyl-C₀₋₆alkyl-. Preferably R² is Ar—C₀₋₆alkyl- or optionally substituted Het-C₀₋₆alkyl-. More preferably R² is Ar—C₁alkyl- or optionally substituted Het-C₁alkyl-. Most preferably R² is benzyl, optionally substituted methylfuranyl or optionally substituted methylthiophenyl.

Suitably, pharmaceutically acceptable salts of formula (I) or formula (IA) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

The compounds of the present invention may contain one or more asymmetric carbon atoms and may exist in racemic and optically active forms. The stereocenters may be (R), (S) or any combination of R and S configuration, for example, (R,R), (R,S), (S,S) or (S,R). All of these compounds are within the scope of the present invention.

Among the preferred compounds of the formula (IA) are the following compounds: 2-(Benzylthio)-4-benzyl-1H-imidazole.

Methods of Preparation:

Compounds of the formula I and formula (IA) are prepared by methods analogous to those described in Scheme 1.

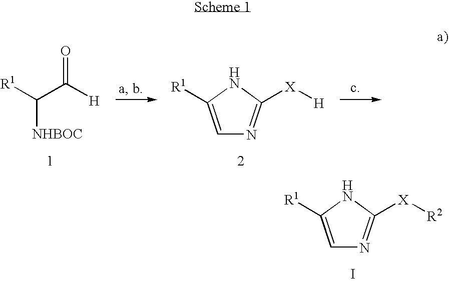

HCl, 1,4-dioxane; b) NH₄SCN or sodium cyanate; c) NaH, R²—CH₂—X, DMF.

An amino-propanal (such as (S)-(−)-2-(tert-butoxycarbonyl-amino)-3-phenylpropanal) was exposed to 4 N HCl in 1,4-dioxane to afford the corresponding amine-hydrochloride. The amine was subsequently treated with ammonium thiocyanate or sodium cyanate to provide the mercapto-imidazole (Heath, H.; Alexander, L., Rimington, C. J. Chem. Soc. 1951, 491). Treatment of the triazole with K₂CO₃ or NaH and an alkyl halide (such as benzyl bromide) in DMF afforded the imidazole.

Formulation of Pharmaceutical Compositions

The pharmaceutically effective compounds of this invention (and the pharmaceutically acceptable salts thereof) are administered in conventional dosage forms prepared by combining a compound of this invention of formula (I) or (IA) (“active ingredient”) in an amount sufficient to treat cancer, haemangiorna, proliferative retinopathy, rheumatoid arthritis, atherosclerotic neovascularization, psoriasis, ocular neovascularization or obesity (“MetAp2-mediated disease states”) with standard pharmaceutical carriers or diluents according to conventional procedures well known in the art. These procedures may involve mixing, granulating and compressing or dissolving the ingredients as appropriate to the desired preparation.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl monostearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1000 mg. When a liquid carrier is used, the preparation will be in the form of a syrup, emulsion, soft gelatin capsule, sterile injectable liquid such as an ampule or nonaqueous liquid suspension.

The active ingredient may also be administered topically to a mammal in need of treatment or prophylaxis of MetAP2-mediated disease states. The amount of active ingredient required for therapeutic effect on topical administration will, of course, vary with the compound chosen, the nature and severity of the disease state being treated and the mammal undergoing treatment, and is ultimately at the discretion of the physician. A suitable dose of an active ingredient is 1.5 mg to 500 mg for topical administration, the most preferred dosage being 1 mg to 100 mg, for example 5 to 25 mg administered two or three times daily.

By topical administration is meant non-systemic administration and includes the application of the active ingredient externally to the epidermis, to the buccal cavity and instillation of such a compound into the ear, eye and nose, and where the compound does not significantly enter the blood stream. By systemic administration is meant oral, intravenous, intraperitoneal and intramuscular administration.

While it is possible for an active ingredient to be administered alone as the raw chemical, it is preferable to present it as a pharmaceutical formulation. The active ingredient may comprise, for topical administration, from 0.001% to 10% w/w, e.g. from 1% to 2% by weight of the formulation although it may comprise as much as 10% w/w but preferably not in excess of 5% w/w and more preferably from 0.1% to 1% w/w of the formulation.

The topical formulations of the present invention, both for veterinary and for human medical use, comprise an active ingredient together with one or more acceptable carrier(s) therefor and optionally any other therapeutic ingredient(s). The carrier(s) must be ‘acceptable’ in the sense of being compatible with the other ingredients of the formulation and not deleterious to the recipient thereof.

Formulations suitable for topical administration include liquid or semi-liquid preparations suitable for penetration through the skin to the site of inflammation such as liniments, lotions, creams, ointments or pastes, and drops suitable for administration to the eye, ear or nose.

Drops according to the present invention may comprise sterile aqueous or oily solutions or suspensions and may be prepared by dissolving the active ingredient in a suitable aqueous or alcoholic solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol. The formulation may incorporate any suitable surface-active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

The active ingredient may also be administered by inhalation. By “inhalation” is meant intranasal and oral inhalation administration. Appropriate dosage forms for such administration, such as an aerosol formulation or a metered dose inhaler, may be prepared by conventional techniques. The daily dosage amount of the active ingredient administered by inhalation is from about 0.1 mg to about 100 mg per day, preferably about 1 mg to about 10 mg per day.

In one aspect, this invention relates to a method of treating cancer, haemangioma, proliferative retinopathy, rheumatoid arthritis, atherosclerotic neovascularization, psoriasis, ocular neovascularization or obesity, all in mammals, preferably humans, which comprises administering to such mammal an effective amount of a MetAP2 inhibitor, in particular, a compound of this invention.

By the term “treating” is meant either prophylactic or therapeutic therapy. Such compound can be administered to such mammal in a conventional dosage form prepared by combining the compound of this invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. The compound is administered to a mammal in need of treatment for cancer, haemangioma, proliferative retinopathy, rheumatoid arthritis, atherosclerotic neovascularization, psoriasis, ocular neovascularization or obesity, in an amount sufficient to decrease symptoms associated with these disease states. The route of administration may be oral or parenteral.

The term parenteral as used herein includes intravenous, intramuscular, subcutaneous, intra-rectal, intravaginal or intraperitoneal administration. The subcutaneous and intramuscular forms of parenteral administration are generally preferred. The daily parenteral dosage regimen will preferably be from about 30 mg to about 300 mg per day of active ingredient. The daily oral dosage regimen will preferably be from about 100 mg to about 2000 mg per day of active ingredient.

It will be recognized by one of skill in the art that the optimal quantity and spacing of individual dosages of a compound of this invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the compound given per day for a defined number of days, can be ascertained by those skilled in the art using conventional course of treatment determination tests.

### EXAMPLES

The invention will now be described by reference to the following examples which are merely illustrative and are not to be construed as a limitation of the scope of the present invention. In the Examples, proton NMR spectra were performed upon a Bruker 400 MHz NMR spectrometer, unless otherwise indicated.

### Example 1

Preparation of 2-(Benzylthio)4-benzyl-1H-imidazole

a) 2-Mercapto-4-benzyl-1H-imidazole

A solution of (S)-(−)-2-(tert-Butoxycarbonyl-amino)-3-phenylpropanal (2.5 g, 10.0 mmol) in 4 N HCl/1,4-dioxane (40 ml) was stirred at room temperature (“rt.”) for 2 h. The crude amine-hydrochloride was concentrated and carried on to the next step. To a solution of the amine-hydrochloride was added ammonium thiocyanate (1.75 g, 23.06 mmol) and the reaction mixture was stirred for 1 h at rt. (Heath, H.; Alexander, L., Rimington, C. J. Chem. Soc. 1951, 491). The reaction mixture was concentrated to provide the title compound as a brown solid (1.85 g, 96%) and was used in the next step without purification. MS (ESI) 191.0 (M+H)⁺.

b) 2-(Benzylthio)-4-benzyl-1H-imidazole

To a stirring solution of 2-mercapto-4-benzyl-1H-imidazole (83 mg, 0.43 mmol) in DMF (1 ml) was added K₂CO₃ (63 mg, 0.46 mmol). The mixture was stirred for 5 min, and benzyl bromide (54 uL, 0.46 mmol) was added via syringe. The mixture was stirred for 15 h at rt., filtered, and the crude imidazole was purified by preparative HPLC to afford the title compound as a white solid (23.4 mg, (33%). MS (ESI) 281. 0 (M)⁺.

Biological Data:

Direct Spectrophotometric Assays of hMetAP2:

The hMetAP2 activity can be measured by direct spectrophotometric assay methods using alternative substrates, L-methionine-p-nitroanilide (Met-pNA) and L-methionine-7-amido-4-methylcoumarin (Met-AMC). The formation of p-nitroaniline (pNA) or 7-amido-4-methylcoumarin (AMC) was continuously monitored by increasing absorbance or fluorescence at 405 nm and 460 nm, respectively, on a corresponding plate reader. All assays were carried out at 30° C. The fluorescence or spectrophotometric plate reader was calibrated using authentic pNA and AMC from Sigma, respectively. For a typical 96-well plate assay, the increase in the absorbance (at 405 nm for pNA) or the fluorescence emission (λₑₓ=360 nm, λₑₘ=460 nm, for AMC) of a 50 μL assay solution in each well was used to calculate the initial velocity of hMetAP2. Each 50 μL assay solution, contained 50 mM Hepes-Na⁺ (pH 7.5), 100 mM NaCl, 10-100 nM purified hMetAP2 enzyme, and varying amounts of Met-AMC (in 3% DMSO aqueous solution) or Met-pNA. Assays were initiated with the addition of substrate and the initial rates were corrected for the background rate determined in the absence of hMetAP2.

Coupled Spectrophotometric Assays of hMetAP2:

The methionine aminopeptidase activity of hMetAP2 can also be measured spectrophotometrically by monitoring the free L-amino acid formation. The release of N-terminal methionine from a tripeptide (Met-Ala-Ser, Sigma) or a tetrapeptide (Met-Gly-Met-Met, Sigma) substrate was assayed using the L-amino acid oxidase (AAO)/horse radish peroxidase (HRP) couple (eq. 1-3a,b). The formation of hydrogen peroxide (H₂O₂) was continuously monitored at 450 nm (absorbance increase of o-Dianisidine (Sigma) upon oxidation, Δε=15,300 M⁻¹cm⁻¹)² and 30° C. in a 96- or 384-well plate reader by a method adapted from Tsunasawa, S. et al.(1997) (eq. 3a). Alternatively, formation of H₂O₂ was followed by monitoring the fluorescence emission increase at 587 nm (Δε=54,000 M⁻¹cm⁻¹, λₑₓ=563 nm, slit width for both excitation and emission was 1.25 mm) and 30° C. using Amplex Red (Molecular Probes, Inc) (Zhou, M. et al. (1997) Anal. Biochem. 253, 162) (eq. 3b). In a total volume of 50 μL, a typical assay contained 50 mM Hepes-Na⁺, pH 7.5, 100 mM NaCl, 10 μM CoCl₂, 1 mM o-Dianisidine or 50 μM Amplex Red, 0.5 units of HRP (Sigma), 0.035 unit of AAO (Sigma), 1 nM hMetAP2, and varying amounts of peptide substrates. Assays were initiated by the addition of hMetAP2 enzyme, and the rates were corrected for the background rate determined in the absence of hMetAP2.

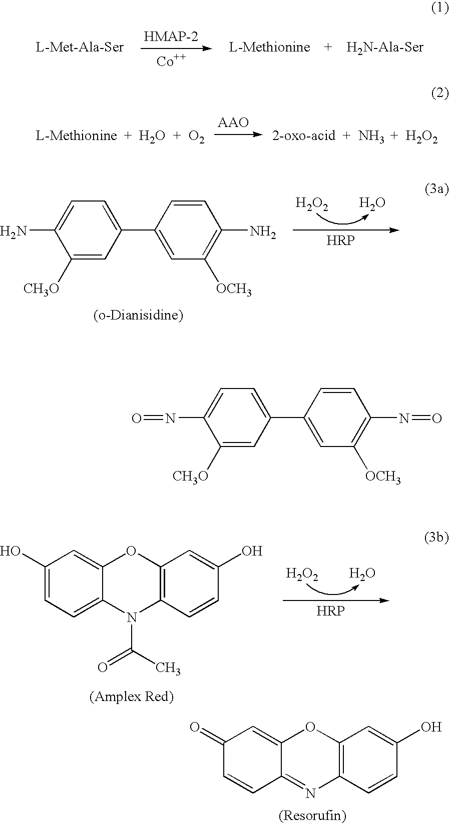

Kinetic Data Analysis:

Data were fitted to the appropriate rate equations using Grafit computer software. Initial velocity data conforming to Michaelis-Menton kinetics were fitted to eq. 4. Inhibition patterns conforming to apparent competitive and non-competitive inhibition were fitted to eq. 5 and eq. 6, respectively.

ν=VA/(Kₐ+A) (4)

ν=VA/[Kₐ(1+I/Kᵢₛ)+A] (5)

ν=VA/[Kₐ(1+I/Kᵢₛ)+A(1+I/Kᵢᵢ)] (6)

In eqs. 4-6, v is the initial velocity, V is the maximum velocity, Kₐ is the apparent Michaelis constant, I is the inhibitor concentration, and A is the concentration of variable substrates. The nomenclature used in the rate equations for inhibition constants is that of Cleland (1963), in which Kᵢₛ and Kᵢᵢ represent the apparent slope and intercept inhibition constants, respectively.

Cell Growth Inhibition Assays:

The ability of MetAP2 inhibitors to inhibit cell growth was assessed by the standard XTT microtitre assay. XTT, a dye sensitive to the pH change of mitochondria in eukaryotic cells, is used to quantify the viability of cells in the presence of chemical compounds. Cells seeded at a given number undergo approximately two divisions on average in the 72 hours of incubation. In the absence of any compound, this population of cells is in exponential growth at the end of the incubation period; the mitochondrial activity of these cells is reflected in the spectrophotometric readout (A450). Viability of a similar cell population in the presence of a given concentration of compound is assessed by comparing the A450 reading from the test well with that of the control well. Flat-bottomed 96-well plates are seeded with appropriate numbers of cells (4-6×10³ cells/well in a volume of 200 ul) from trypsinized exponentially growing cultures. In the case of HUVECs, the wells are coated with matrigel prior to establishing the cultures. To “blank” wells is added growth medium only. Cells are incubated overnight to permit attachment. Next day, medium from wells that contain cells is replaced with 180 ul of fresh medium. Appropriate dilutions of test compounds are added to the wells, final DMSO concentration in all wells being 0.2%. Cells plus compound are incubated for an additional 72 hr at 37° C. under the normal growth conditions of the cell line used. Cells are then assayed for viability using standard XTT/PMS (prepared immediately before use: 8 mg XTT (Sigma X-4251) per plate is dissolved in 100 ul DMSO. 3.9 ml H₂O is added to dissolve XTT and 20 ul of PMS stock solution (30 mg/ml) is added from frozen aliquoted stock solution (10 mg of PMS (phenazine methosulfate, Sigma P-9625) in 3.3 ml PBS without cations. These stocks are frozen at −20° C. until use). 50 ul of XTT/PMS solution is added to each well and plates incubated for 90 minutes (time required may vary according to cell line, etc.) at 37° C. until A₄₅₀ is >1.0. Absorbance at 450 nM is determined using a 96-well UV plate reader. Percent viability of cells in each well is calculated from these data (having been corrected for background absorbance). IC50 is that concentration of compound that reduces cell viability to 50% control (untreated) viability.

The compounds of this invention show MetAP2 inhibitor activity having IC₅₀ values in the range of 0.0001 to 100 uM. The full structure/activity relationship has not yet been established for the compounds of this invention. However, given the disclosure herein, one of ordinary skill in the art can utilize the present assays in order to determine which compounds of this invention are inhibitors of MetAP2 and which bind thereto with an IC₅₀ value in the range of 0.0001 to 100 uM.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A method of inhibiting MetAP2 in mammals, comprising administering to a mammal in need of such treatment, an effective amount of a compound of formula (IA) or a pharmaceutically acceptable salt or solvate thereof: 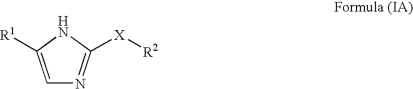
wherein:
X is S or O;
R¹ is optionally substituted C₁₋₆alkyl, C₂₋₆alkenyl, C₂₋₆alkynyl, optionally substituted Ar—C₀₋₆alkyl-, optionally substituted Het-C₀₋₆alkyl-, or optionally substituted C₃₋₇cycloalkyl-C₀₋₆alkyl-; and
R² is optionally substituted C₁₋₆alkyl, C₃₋₆alkenyl, C₃₋₆alkynyl, optionally substituted Ar—C₀₋₆alkyl-, optionally substituted Het-C₀₋₆alkyl-, or optionally substituted C₃₋₇cycloalkyl-C₀₋₆alkyl-.

2. The method of claim 1, wherein the compound of formula (IA) is 2-(benzylthio)-4-benzyl-1H-imidazole, or a pharmaceutically acceptable salt or solvate thereof.

3. A method for treating a disease mediated by MetAP2 in mammals, comprising administering to a mammal in need of such treatment, an effective amount of a compound of formula (IA) or a pharmaceutically acceptable salt or solvate thereof: 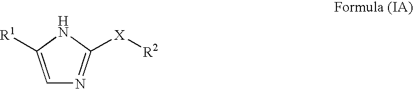
wherein:
X is S or O;
R¹ is optionally substituted C₁₋₆alkyl, C₂₋₆alkenyl, C₂₋₆alkynyl, optionally substituted Ar—C₀₋₆alkyl-, optionally substituted Het-C₀₋₆alkyl-, or optionally substituted C₃₋₇cycloalkyl-C₀₋₆alkyl-; and
R² is optionally substituted C₁₋₆alkyl, C₃₋₆alkenyl, C₃₋₆alkynyl, optionally substituted Ar—C₀₋₆alkyl-, optionally substituted Het-C₀₋₆alkyl-, or optionally substituted C₃₋₇cycloalkyl-C₀₋₆alkyl-.

4. The method of claim 3, wherein the compound of formula (IA) is 2-(benzylthio)-4-benzyl-1H-imidazole, or a pharmaceutically acceptable salt or solvate thereof.

5. A compound of formula (1), or a pharmaceutically acceptable salt or solvate thereof: 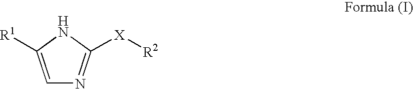
wherein:
X is S or O;
R¹ is optionally substituted C₁₋₆alkyl, C₂₋₆alkenyl, C₂₋₆alkynyl, optionally substituted Ar—C₀₋₆alkyl-, optionally substituted Het-C₀₋₆alkyl-, or optionally substituted C₃₋₇cycloalkyl-C₀₋₆alkyl-; provided that when R¹ is optionally substituted Het-C₁₋₄alkyl-, and Het is indolyl, benzofuranyl, benzothienyl, benzisoxazolyl, benzisothiazolyl, benzopyrazolyl, or pyrrolo[2,3-c]pyridinyl then the optional substituent is not CH₂)₁₋₅CHR^{I}NR^{II}R^{III}, or the optional substitutent is not a 4- to 6-membered heterocycle which contains one nitrogen; or provided that when R¹ is Ar—C₁₋₂alkyl-, Ar is phenyl optionally substituted at the meta or para position with —CN, —C(═NR)NR′R″, —NHC(═NR)R′R″, —NRC═NR, or —CONRR′, wherein R, R′ and R″ are independently H, C₁₋₆alkyl, C₂₋₆alkenyl, C₂₋₆alkynyl, Ar—C₀₋₆alkyl-, Het-C₀₋₆alkyl-, or C₃₋₇cycloalkyl-C₀₋₆alkyl-; or provided that when R¹ is optionally substituted Ar—C₁alkyl-, the optional substituents are not both —OH and phenyl or a saturated 6-membered ring containing one nitrogen;
R^{I} is H or C₁₋₆alkyl;
R^{II} and R^{III} are independently H, C₁₋₆alkyl, or together with the nitrogen to which they are attached form a 4-6 membered heterocyclic ring which optionally contains one or more additional heteroatoms selected from N, O, and S; and
R² is optionally substituted C₁₋₆alkyl, C₃₋₆alkenyl, C₃₋₆alkynyl, optionally substituted Ar—C₀₋₆alkyl-, optionally substituted Het-C₀₋₆alkyl-, or optionally substituted C₃₋₇cycloalkyl-C₀₋₆alkyl-; provided that when R¹ is optionally substituted Ar—C₀alkyl-, or optionally substituted C₅₋₆cycloalkyl-C₀alkyl- then R² is not C₁₋₆alkyl; or provided when R¹ is optionally substituted Ar—C₀alkyl-, or C₅₋₆cycloalkyl-C₀alkyl- R² is not imidazolyl-C₂₋₃alkyl-, or provided that the compound is not 2-[[(4-phenyl-1H-imidazol-2-yl)thio]methyl]-pyridinyl, 4-phenyl-2-(benzylthio)-1H-imidazole, 4-phenyl-2-(4-chloro-benzylthio)-1H-imidazole, 4-phenyl-2-(2-methylamino-benzylthio)-1H-imidazole, 4-phenyl-2-(2-propenylthio)-1H-imidazole, 2-[[4-(3-thienyl)-1H-imidazol-2-yl]thio]-hexanoic acid, 4-phenyl-2-(phenylthio)-1H-imidazole, 4-cyclohexyl-2-[(2-methyl-2-propenyl)thio]-1H-imidazole, 4-(1-methylcyclohexyl)-2-[(2-methyl-2-propenyl)thio]-1H-imidazole, 2,6-bis(1,1-dimethylethyl)-4-[2-[(phenylmethyl)thio]-1H-imidazole, 2-[[[4-(4-methoxyphenyl)-1H-imidazol-2-yl]thio]methyl]-pyridinyl, 2-[[[4-(4-bromophenyl)-1H-imidazol-2-yl]thio]methyl]-pyridinyl, or 1-(cyclopropylamino)-3-[[4-(2-thienyl)-1H-imidazol-2-yl]oxy]-2-propanol dihydrochloride.

6. A pharmaceutical composition comprising a compound as claimed in claim 5 and a pharmaceutically acceptable carrier.

7. A compound of formula (I) which is 2-(benzylthio)-4-benzyl-1H-imidazole.
